(19) United States
(12) Patent Application Publication
(10) Pub. No.: US 2010/0144729 A1
(43) Pub. Date: Jun. 10, 2010

(54) COUMARIN DERIVATIVES

(76) Inventors: Rolf Baenteli, Zurich (CH); Nigel Graham Cooke, Oberwil (CH); Frederic Zecri, Bartenheim (FR); Alexander Baxter Smith, Niffer (FR)

Correspondence Address: Fanelli Strain & Haag PLLC, 1455 Pennsylvania Ave., N.W., suite 400, Washington, DC, 20004, US

(21) Appl. No.: 12/524,943

(22) PCT Filed: Jan. 31, 2008
(86) PCT No.: PCT/EP08/51218

§ 371 Date: Jul. 29, 2009

(30) Foreign Application Priority Data
Feb. 2, 2007 (EP) 07101669.5
Oct. 11, 2007 (EP) 07118310.7

(51) Int. Cl. A61K 31/5377; C07D 311/02; A61K 31/366; C07D 405/12; A61K 31/4025; A61K 31/453; C07D 413/12

(52) U.S. Cl. 514/233.5; 549/287; 514/456; 548/525; 514/422; 514/320; 546/196; 544/151

(57) ABSTRACT

A compound of formula (I) wherein R₁, R₂, R₃ and R₄ are as defined in the specification, processes for their production, their uses, in particular in transplantation, and pharmaceutical compositions containing them.

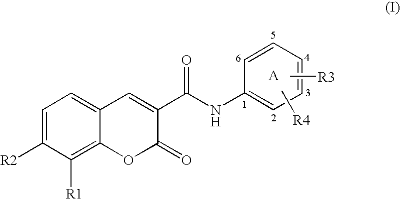

The present invention relates to polycyclic compounds, processes for their production, their use as pharmaceuticals and to pharmaceutical compositions comprising them.

More particularly the present invention provides in a first aspect a compound of formula I

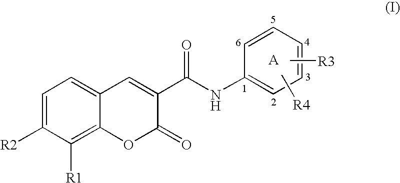

wherein
each of R₁ and R₂, independently, is selected from the group consisting of hydrogen; halogen; nitro; optionally substituted C₁₋₈alkyl (e.g. substituted by aryl, C₃₋₆cycloalkyl, or C₁₋₈alkoxy); optionally substituted haloC₁₋₈alkyl; optionally substituted C₁₋₈alkoxy (e.g. substituted by C₁₋₈alkoxy, C₃₋₈cycloalkyl, aryl); optionally substituted haloC₁₋₈alkoxy;

R₃ is a saturated heterocyclic ring comprising at least one ring N atom and which is attached to ring A through a ring C atom of R₃, this or any other ring C atom being optionally substituted (e.g. by halogen, C₁₋₆alkyl, C₁₋₆alkoxy, C₁₋₆alkylC₁₋₆alkoxy); and

R₃ is in position 3 or 4 of said ring A, preferably in position 4;

R₄ is hydrogen; hydroxyl; halogen; haloC₁₋₈alkyl; optionally substituted C₁₋₆alkyl; C₁₋₆alkoxy; or haloC₁₋₆alkoxy; and

R₄ is in position 2 (ortho) or 3 (meta);

or a physiologically hydrolysable derivative thereof, a salt, hydrate and/or solvate thereof.

Halogen may be fluorine, chlorine or bromine, preferably fluorine or chlorine.

Alkyl or alkoxy as a group or present in a group may be straight or branched. Alkylene may be straight or branched.

Alkyl as a group or present in a group may be substituted, e.g. by hydroxyl, halogen, alkoxy.

Alkoxy as a group or present in a group may be substituted, e.g. by hydroxyl.

When alkyl or alkoxy is substituted by hydroxyl, the hydroxyl group is preferably at the terminal position of the alkyl or alkoxy.

Alkenyl may be substituted e.g. by alkyl, hydroxyl.

As herein defined haloalkyl and haloalkoxy refers to alkyl and alkoxy, respectively, either as a group or present in a group, which is substituted by 1 to 5 halogen, e.g. CF₃—, CHF₂—, CH₂F— or CF₃—CH₂—O—, CHF₂—CH₂—O—, CH₂F—CH₂—O—.

Haloalkyl and haloalkoxy may be substituted e.g. by hydroxyl.

Any aryl may be phenyl or naphthyl, preferably phenyl.

By C₃₋₈cycloalkyl, as a group or present in a group, e.g. as R₁, R₂, is meant a three to eight, preferably five to seven, membered non aromatic ring, comprising no heteroatom.

Preferably R₃ is of formula (Ia)

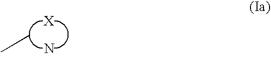

wherein X is selected from N, O, C and S atom.

Preferably R₃ is a three to eight membered saturated, heterocyclic ring of formula (Ib)

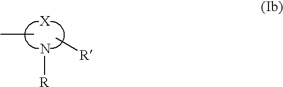

wherein X is selected from NR, O, CH₂ and S atom, wherein

R and R′ are independently from each other H or C₁ alkyl, and

wherein said R₃ is attached to said ring A in formula (I) via any carbon atom within the ring of formula (Ib) carrying a hydrogen atom, which hydrogen is then replaced by said attachment.

In the formula (Ib) the variables can have the following preferred meanings independently, collectively or in any combination thereof:

R is H, methyl or ethyl;

R′ is H or methyl;

X is selected from NR, O, and CH₂ wherein R is H or C₁₋₄ alkyl or more preferably H, methyl or ethyl;

residue of formula (Ib) may be chiral, e.g. R- or S-enantiomer, or may be a racemic mixture thereof.

In a still further aspect the present invention relates to a metabolite of the compounds of formula (I), for example to a compound of formula (I), wherein R₃ is a residue of formula (Ic) or (Id)

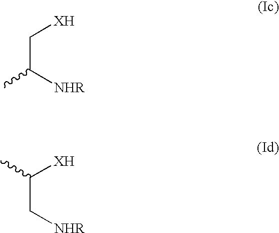

wherein X is selected from O, and S atom,

R is H or C₁₋₄ alkyl, and

the wavy bond denotes the R— or S— enantiomer or the mixture thereof.

For example, R₃ is a three to eight, preferably five to eight, membered saturated, heterocyclic ring comprising 1 or 2 heteroatoms, preferably selected from N, O and S. It will be appreciated by the man of skill that 2 heteroatoms may typically be present in a heterocyclic ring when the ring size is from five to eight atoms. Consequently in a three and/or four membered heterocyclic ring no more than 1 heteroatom is typically present. In a particular aspect, R₃ is attached to ring A through the ring C atom of R₃ which is in position ortho or meta vis a vis the ring N atom of R₃ (or vis a vis one of the ring N atoms of R₃).

The preferred R₃ is selected from 3-morpholinyl, 2-morpholinyl, 2-piperazinyl, 2-piperidinyl, 3-piperidinyl, 3-thiomorpholinyl, 2-thiomorpholinyl, 2-pyrrolidinyl, and 3-pyrrolidinyl.

Optionally R₃ is substituted. When R₃ is substituted, this may be on one or more ring carbon atoms and/or on a ring nitrogen atom when present. Examples of a substituent on a ring carbon atom include e.g. C₁₋₄alkyl, C₁₋₆alkoxy, halogen, halogenoC₁₋₄alkyl. Examples of a substituent on a ring heteroatom include e.g. C₁₋₄alkyl.

Ring A comprises no heteroatom.

The following significances are preferred independently, collectively or in any combination or sub-combination:
1. each of R₁ and R₂, independently, is selected from the group consisting of hydrogen; optionally substituted C₁₋₈alkyl; optionally substituted haloC₁₋₈alkyl; optionally substituted C₁₋₈alkoxy; optionally substituted haloC₁₋₈alkoxy;
2. each of R₁ and R₂, independently, is selected from the group consisting of hydrogen; C₁₋₈alkyl; C₁₋₈alkoxy;
3. R₁ and R₂ do not both represent hydrogen;
4. R₁ and R₂ are both C₁₋₈alkoxy;
5. R₃ is 3-morpholinyl, 2-piperazinyl, 2-piperidinyl or 2-pyrrolidinyl;
6. R₃ is 3-S-morpholinyl, 2-S-piperazinyl, 2-R-piperidinyl or 2-R-pyrrolidinyl;
7. R₃ is in position 4;
8. each of R₁ and R₂, independently, is selected from the group consisting of hydrogen; C₁₋₈alkyl; C₁₋₈alkoxy; R₃ is selected from the group consisting of 3-morpholinyl, 2-piperazinyl, 2-piperidinyl, and 2-pyrrolidinyl;
9. each of R₁ and R₂, independently, is selected from the group consisting of C₁₋₈alkyl; C₁₋₈alkoxy; R₃ is selected from the group consisting of 3-S-morpholinyl, 2-S-piperazinyl, 2-R-piperidinyl, and 2-R-pyrrolidinyl; and R₄ is hydrogen.
10. each of R₁ and R₂, independently, is selected from the group consisting of optionally substituted C₁₋₈alkyl; optionally substituted haloC₁₋₈alkyl; optionally substituted C₁₋₈alkoxy; and optionally substituted haloC₁₋₈alkoxy;
11. each of R₁ and R₂, independently, is selected from the group consisting of C₁₋₈alkyl; haloC₁₋₈alkyl; C₁₋₈alkoxy; and haloC₁₋₈alkoxy;
12. each of R₁ and R₂, independently, is selected from the group consisting of C₁₋₈alkyl; and C₁₋₈alkoxy;
13. R₃ is a chiral residue and is an R- or S-enantiomer or a mixture thereof (racemate);
14. R₃ is a S-enantiomer;
15. R₃ is a R-enantiomer;
16. R₄ is hydrogen

The compounds of formula I may exist in free form or in salt form, e.g. addition salts with e.g. organic or inorganic acids, for example, hydrochloric acid or acetic acid, or salts obtainable when R5 is or comprises COOH, with a base, e.g. alkali salts such as sodium or potassium, or substituted or unsubstituted ammonium salts.

It will be appreciated that the compounds of formula I may exist in the form of optical isomers, racemates or diastereoisomers. It is to be understood that the present invention embraces all enantiomers and conformers and their mixtures. Similar considerations apply in relation to starting materials exhibiting asymmetric carbon atoms as mentioned above.

By a physiologically hydrolysable derivative of a compound of formula I is meant a compound which is hydrolysable under physiological conditions to yield a compound of formula I and a by-product which is itself physiologically acceptable, e.g. an ester which is hydrolyzed to yield a compound of formula I and a non-toxic alcohol at the desired dosage levels.

The present invention also includes processes for the production of a compound of formula I, which processes comprise reacting a compound of formula II, wherein R₁ and R₂ are as defined above with a compound of formula III, wherein R₃ and R₄ are as defined above as shown in route A (scheme 1).

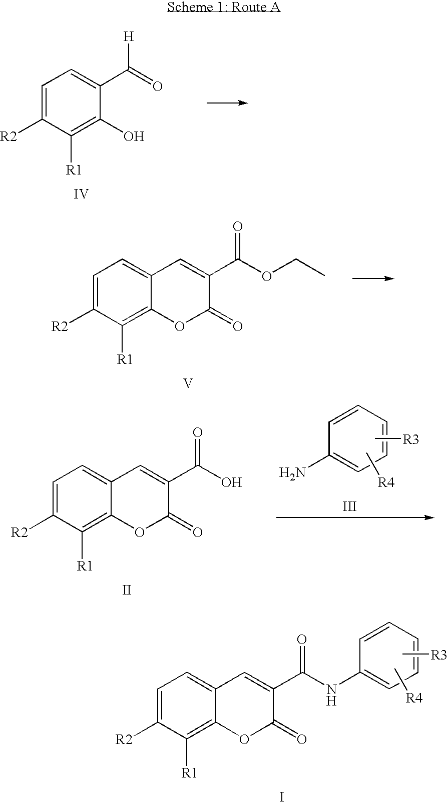

All reactions are performed in a solvent such as methanol, ethanol, tetrahydrofuran, toluene, dichloromethane, 1,2-dichloroethane, N-methylpyrolidone, xylenes, ethyl acetate, diethyl ether, hexanes, cyclohexanes, dimethylformamide, acetone, dimethylsulfoxide, tert-butylmethyl ether. All compounds can be isolated using methods known to those skilled in the art (e.g. crystallization, silica gel chromatography, HPLC).

2-hydroxy benzaldehydes of formula IV are condensated with diethyl malonate in the presence of a suitable base (for example a secondary amine such as piperidine) in a suitable solvent to give 2-oxo-2H-chromene-3-carboxylic ester of formula V. In case, R2 is equal to hydroxy, at this stage the hydroxyl group can be alkylated to give R2 equals alkoxy under basic conditions using an alkylhalide as electrophile in presence of a suitable base seach as triethyl amine, piperidine, sodium hydride, potassium carbonate or cesium carbonate in presence of a suitable solvent, or using Mitsunobu conditions with the corresponding alcohol in presence of triphenyl phosphine and DEAD reagent.

2-Oxo-2H-chromene-3-carboxylic esters of formula V are then saponified in presence of a lithium hydroxide or sodium hydroxide in a suitable solvent to give 2-oxo-2H-chromene-3-carboxylic acids of formula II.

Compounds of formula II are activated for amide bond formation with a reagent such as thionyl chloride or 1-(3-dimethylaminopropyl)-3-ethylcarbodiimide or 1,1′-carbonyldiimidazole or propanephosphonic anhydride in the presence of a suitable base such as triethyl amine, N,N-diisopropylethylamine or sodium bicarbonate in a suitable solvent and reacted with a compound of formula III (aniline derivative) leading to the desired compound of formula I. If R₄ or R₅ contains a nitrogen functionality protecting group e.g. a carbamic acid tert-butyl ester function, deprotection is effected by reacting it with an acid such as hydrochloric acid or trifluoroacetic acid in a suitable solvent.

Methods to prepare 2-oxo-2H-chromene-3-carboxylic acid and compounds of formula I as described in route A as well as other methods pertinent to the present invention are known to the one skilled in the art and have been reviewed in the literature (Horing, E. C. et al. (1955) organic synthesis, Coll. Vol. III, 165, Livingstone, R. (1977); Rodd's Chemistry of carbon compounds, Vol. IV, p 96, Staunton, J. (1979); Heterocyclic Chemistry (ed. P. G. Sammes), Vol. 4).

Insofar as the production of the starting materials is not particularly described, the compounds are either known or may be prepared analogously to methods known in the art or as disclosed hereinafter.

A convenient method to prepare non-commercial 2-hydroxy benzaldehyde compounds of formula IV wherein R₁ is allyl or propyl is shown in scheme 2 (route B). 2-Hydroxy benzaldehydes of formula IV wherein R₁ is H can be O-alkylated with an electrophile such as allylbromide in presence of a suitable base such as potassium carbonate or cesium carbonate in a suitable solvent to give compound of formula VI. Claisen rearrangement of compounds of formula VI under thermic conditions (oil bath or microwave heating) can be carried out neat or in a suitable solvent to obtain compounds of formula IV wherein R₁ is allyl. Selective reduction of the double bond in presence of the aldehyde to give compounds of formula IV wherein R₁ is propyl can be achieved under standard hydrogenation conditions using Raney Nickel as a catalyst in a suitable solvent.

Alternatively as shown in scheme 2 (route C) if R₂ is alkoxy, compounds of formula VII are reacted with a strong base such as butyl lithium and an alkyl halide or an acyl halide to give compounds of formula VIII, which are O-dealkylated by the action of an acid such as hydrochloric acid, hydrobromic acid or boron tribromide in a suitable solvent to give compounds of formula IX (phenols). Compounds of formula X are converted into compounds of formula IV wherein R1 is alkyl, or —COalkyl, and R2 is OH under Vilsmeier conditions using for example POCl₃ and N,N-dimethylformamide as a carbonyl source in a suitable solvent.

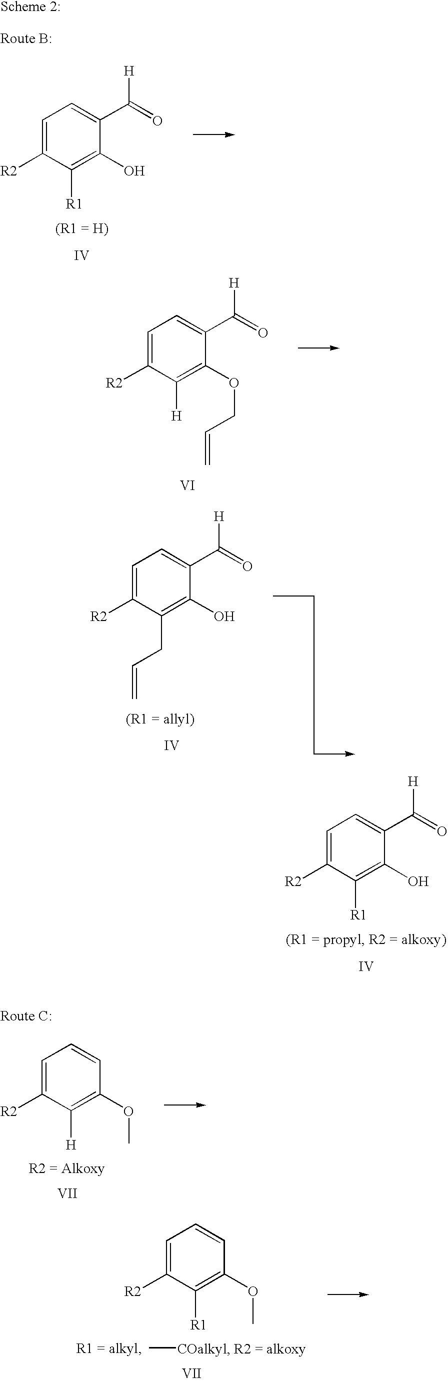

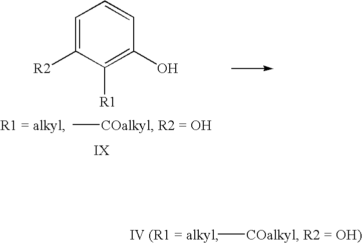

A convenient method to prepare non-commercial 2-hydroxy benzaldehyde intermediates of formula IV wherein R2 is hydroxy is shown in scheme 3 (route D). The synthesis of compounds of formula XI is reproduced according to a literature procedure (Synthetic Communications, 20(12), 1869-1876). Compounds of formula X are converted into compounds of formula IV wherein R2 is hydroxy under Vilsmeier conditions using for example POCl₃ and N,N-dimethylformamide as a carbonyl source in a suitable solvent.

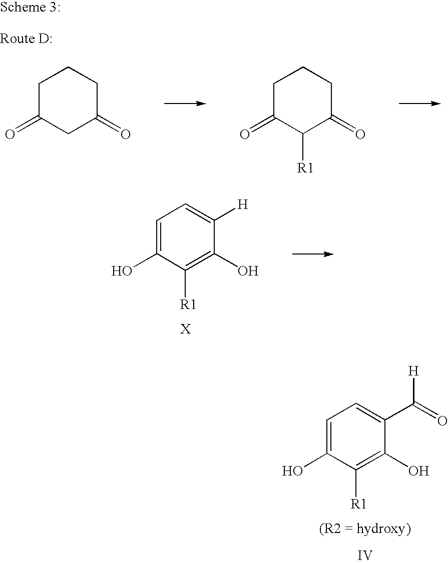

Aniline intermediates of formula III can be purchased or the respective nitro compounds are purchased and reduced to the anilines of formula III by the action of palladium on charcoal and hydrogen or palladium on charcoal with sodiumborohydride or tindichloride in a suitable solvent. If R₄ or R₅ is bearing an amino function this is protected as a tert-butoxycarbamate using BOC anhydride as an electrophile in presence of a suitable base such as triethyl amine, diethyl isopropyl amine in a suitable solvent.

Anilines of formula III can be obtained from the corresponding nitro or bromo phenyl XI by either reduction by the action of palladium on charcoal and hydrogen or palladium on charcoal with sodiumborohydride or tindichloride in a suitable solvent or by bromo to amino exchange under standard Pd catalysed conditions using nitrogen source such as ammonia, hexamethyldisilazane or iminobenzophenone. Nitro or bromo phenyl of formula XI can be obtained under standard nitration or bromination conditions from the corresponding phenyl compound of formula XII as outlined in scheme 4 (route E).

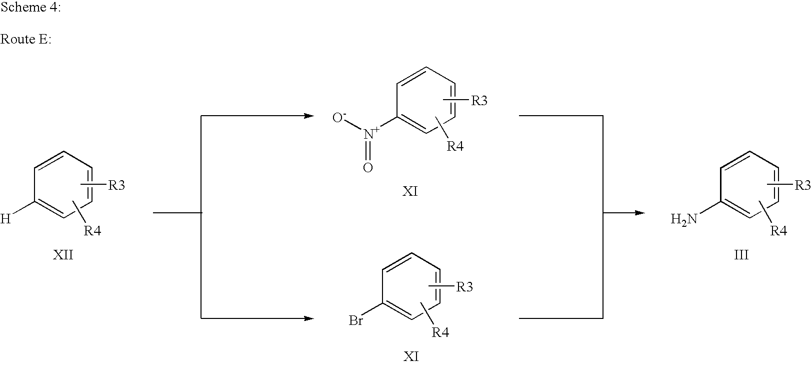

The following examples are illustrative of the invention.

Concentration of solutions is carried out on a rotary evaporator under reduced pressure. Conventional flash chromatography is carried out on silica gel. Flash chromatography is also carried out using Biotage Flash Chromatography apparatus or Flashmaster instrument.

### Abbreviations Used are:

TBME=tert-butylmethyl ether
BOC=tert-butyloxy carbonyl
DMF=dimethylformamide
LiOH=lithium hydroxide
HCl=hydrochloric acid
THF=tetrahydrofuran
CH₂Cl₂=dichloromethane
RT=room temperature
NaOH=sodium hydroxide

Min=minute

### EXAMPLE 1

### 7-Methoxy-2-oxo-8-propyl-2H-chromene-3-carboxylic acid (4-pyrrolidin-2-yl-phenyl)-amide

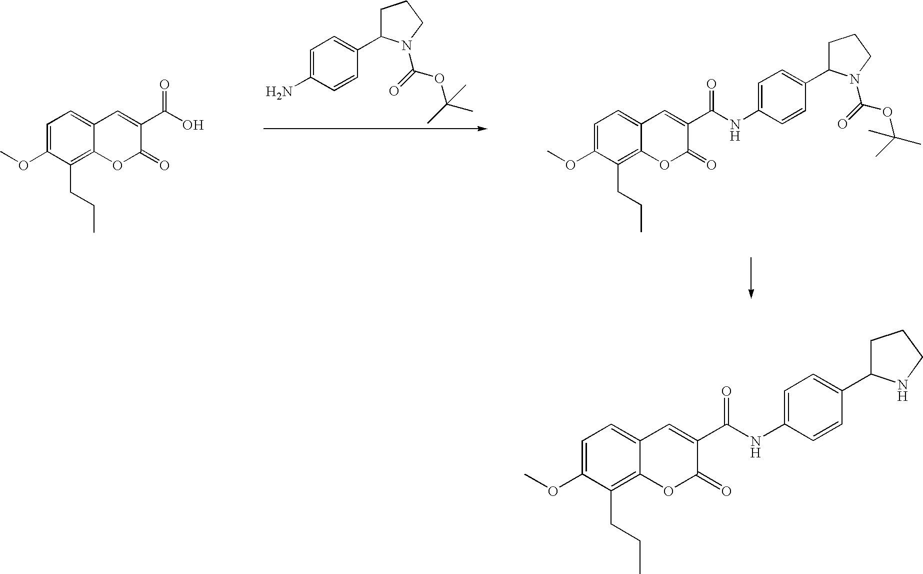

### a) Preparation of 2-allyloxy-4-methoxy-benzaldehyde

To a solution of 2-hydroxy-4-methoxy-benzaldehyde (5 g, 32.8 mmol) and allyl bromide (3.89 ml, 46 mmol) in acetone (50 ml) is added potassium carbonate (6.8 g, 49.3 mmol). The reaction mixture is then stirred under reflux for 3 hours. The reaction mixture is concentrated and partitioned between 200 ml of TBME and 150 ml of 1N NaOH and the layers were separated. The organic layer is washed with 150 ml of brine and 150 ml of water, dried and concentrated. 2-allyloxy-4-methoxy-benzaldehyde is isolated after purification using flash chromatography (eluent CH₂Cl₂/Hexanes 8/2).

### b) Preparation of 3-allyl-2-hydroxy-4-methoxy-benzaldehyde

A solution of 2-allyloxy-4-methoxy-benzaldehyde (5 g, 26 mmol) in NMP (10 ml) is microwave heated at 230° C. for 30 minutes. The reaction mixture is then poured into an ice/water (200 ml) mixture and TBME (200 ml) is added, the organic layer is separated and washed with 150 ml of brine and 150 ml of water, dried and concentrated. 3-Allyl-2-hydroxy-4-methoxy-benzaldehyde is isolated after purification using flash chromatography (eluent CH₂Cl₂/Hexanes 8/2).

### c) Preparation of 2-hydroxy-4-methoxy-3-propyl-benzaldehyde

To a solution of 3-allyl-2-hydroxy-4-methoxy-benzaldehyde (5 g, 26 mmol) in THF (25 ml) is added 10% wt Pt/C. The reaction mixture is then stirred at room temperature until 1 eq of hydrogen gas is consumed. The reaction mixture is then filtered over celite and concentrated. 2-hydroxy-4-methoxy-3-propyl-benzaldehyde is used without further purification.

### d) Preparation of 7-methoxy-2-oxo-8-propyl-2H-chromene-3-carboxylic acid ethyl ester

To a solution of 2-hydroxy-4-methoxy-3-propyl-benzaldehyde (15 g, 77.2 mmol) in ethanol (450 ml) is added diethyl malonate (11.7 ml, 77.2 mmol) and piperidine (7.6 ml, 77.2 mmol). The reaction mixture is stirred at RT overnight. The reaction mixture is then cooled to 0° C. using a ice/water bath and the formed precipitate is filtered and washed with ethanol. The mother liquor are concentrated and purified using flash chromatography (eluent ethyl acetate/Hexanes 3/7) to yield 7-methoxy-2-oxo-8-propyl-2H-chromene-3-carboxylic acid ethyl ester.

### e) Preparation of 7-methoxy-2-oxo-8-propyl-2H-chromene-3-carboxylic acid

To a solution of 7-methoxy-2-oxo-8-propyl-2H-chromene-3-carboxylic acid ethyl ester (15.4 g, 53.0 mmol) in THF (300 ml) is added at 0° C. a 1N solution of NaOH (120 ml), the reaction mixture is then stirred overnight at RT. The reaction mixture is cooled to 0° C. using an ice/water bath and the pH was brought down to 1 using a 1N HCl solution. The reaction mixture is stirred at 0° C. for 30 minutes and the formed precipitate is filtered and washed with water. 7-methoxy-2-oxo-8-propyl-2H-chromene-3-carboxylic is isolated after drying the precipitate.

### f) Preparation of 2-{4-[(7-Methoxy-2-oxo-8-propyl-2H-chromene-3-carbonyl)-amino]-phenyl}-pyrrolidine-1-carboxylic acid tert-butyl ester

To a solution of 7-methoxy-2-oxo-8-propyl-2H-chromene-3-carboxylic acid (150 mg, 0.572 mmol) in dichloromethane (6 ml) is added diisopropylethyl amine (147 uL, 0.858 mmol) and a solution of propylphosphonic anhydride in ethyl acetate (456 uL, 0.858 mmol). The reaction mixture is then stirred at room temperature for 30 minutes followed by addition of 2-(4-amino-phenyl)-pyrrolidine-1-carboxylic acid tert-butyl ester (150 mg, 0.572 mmol). The reaction mixture is then stirred at room temperature for 4 hours The reaction mixture is diluted with dichloromethane (40 ml) and is washed with 40 ml of saturated solution of NaHCO₃, 40 ml of brine, the organic layer is then extracted, dried and concentrated. 2-{4-[(7-Methoxy-2-oxo-8-propyl-2H-chromene-3-carbonyl)-amino]-phenyl}-pyrrolidine-1-carboxylic acid tert-butyl ester is isolated after precipitation and washes using hexanes to afford a slightly yellow solid.

### g) Preparation of 7-methoxy-2-oxo-8-propyl-2H-chromene-3-carboxylic acid (4-pyrrolidin-2-yl-phenyl)-amide

To a solution of 2-{4-[(7-methoxy-2-oxo-8-propyl-2H-chromene-3-carbonyl)-amino]-phenyl}-pyrrolidine-1-carboxylic acid tert-butyl ester (194 mg, 0.38 mmol) in dichloromethane (5 ml) is added 4N HCl in diovane (1 mL). The reaction mixture is stirred at room temperature for 2 h (precipitate forms after addition of HCl). The reaction mixture is then concentrated under reduced pressure and the solid triturated with diethyl ether. 7-Methoxy-2-oxo-8-propyl-2H-chromene-3-carboxylic acid (4-pyrrolidin-2-yl-phenyl)amide is isolated after filtration.

All the following examples are synthesized accorded to the procedure described above for.

By following the procedure as described in above Example 1 and using methods described in Route A, and using the appropriate starting materials the compounds of formula Xa

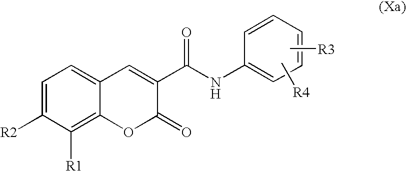

wherein R₁, R₂, R₃ and R₄ are as defined in Table 1 below, are obtained.

TABLE 1

| Ex | R1 | R2 | R3 | R4 | (M + H)⁺ |
|---|---|---|---|---|---|
| 2 | Propyl | OEt | rac-4-pyrrolidin-2-yl- | H | 421.3 |
| 3 | Propyl | OMe | 4-pyrrolidin-2-yl-* | H | 407.3 |
| 4 | Propyl | OMe | 4-pyrrolidin-2-yl-* | H | 407.4 |
| 5 | Ethyl | OMe | 4-pyrrolidin-2-yl-* | H | 393.3 |
| 6 | Ethyl | OMe | 4-pyrrolidin-2-yl-* | H | 393.3 |
| 7 | iPr | OMe | 4-pyrrolidin-2-yl-* | H | 407.3 |
| 8 | OEt | OMe | 4-(R)-pyrrolidin-2-yl- | H | 409.1 |
| 9 | OEt | OMe | 4-pyrrolidin-2-yl-* | H | 409.1 |
| 10 | OiPr | OMe | 4-pyrrolidin-2-yl-* | H | 423.8 |
| 11 | Propyl | OMe | 4-1-methyl-pyrrolidin-2-yl-* | H | 421.4 |
| 12 | Propyl | OMe | 4-1-methyl-pyrrolidin-2-yl-* | H | 421.4 |
| 13 | Propyl | OMe | rac-4-(1-methyl-pyrrolidin-2- | H | 421.3 |
| | | | yl)- | | |
| 14 | Ethyl | OMe | 4-1-methyl-pyrrolidin-2-yl-* | H | 407.4 |
| 15 | Ethyl | OMe | 4-1-methyl-pyrrolidin-2-yl-* | H | 407.4 |
| 16 | Propyl | OEt | rac-4-(1-methyl-pyrrolidin-2- | H | 435.3 |
| | | | yl)- | | |
| 17 | Propyl | OMe | 4-[1-(2-hydroxy-ethyl)- | H | 451.4 |
| | | | pyrrolidin-2-yl]-* | | |
| 18 | iPropyl | OMe | 4-1-methyl-pyrrolidin-2-yl-* | H | 421.3 |
| 19 | Propyl | OMe | rac-4-piperidin-2-yl- | H | 421.3 |
| 20 | Ethyl | OMe | rac-4-piperidin-2-yl- | H | 407.3 |
| 21 | OEt | OMe | 4-piperidin-2-yl-* | H | 423.1 |
| 22 | OEt | OMe | 4-piperidin-2-yl-* | H | 423 |
| 23 | OiPr | OMe | 4-piperidin-2-yl-* | H | 437 |
| 24 | OiPr | OMe | 4-piperidin-2-yl-* | H | 437 |
| 25 | Propyl | OMe | rac-4-(1-methyl-piperidin-2- | H | 435.4 |
| | | | yl)- | | |
| 26 | Ethyl | OMe | rac-4-(1-methyl-piperidin-2- | H | 421.3 |
| | | | yl)- | | |
| 27 | Propyl | OMe | 4-(S)-morpholin-3-yl- | H | 423.2 |
| 28 | Ethyl | OMe | 4-(S)-morpholin-3-yl- | H | 409.1 |
| 29 | OEt | OMe | 4-(S)-morpholin-3-yl- | H | 425.1 |
| 30 | OEt | OMe | 4-(R)-morpholin-3-yl- | H | 425 |
| 31 | OiPr | OMe | 4-(S)-morpholin-3-yl- | H | 439.2 |
| 32 | OCH₂cyclopropyl | OMe | 4-(S)-morpholin-3-yl- | H | 451 |
| 33 | OEt | OMe | 4-(S)-4-methyl-morpholin-3-yl- | H | 439.8 |
| 34 | Methyl | OMe | rac-4-morpholin-2-yl- | H | 395.1 |
| 35 | Ethyl | OMe | rac-4-morpholin-2-yl- | H | 409.5 |
| 36 | Ethyl | OMe | 4-morpholin-2-yl-* | H | 409.1 |
| 37 | OEt | OMe | rac-4-morpholin-2-yl- | H | 425.1 |
| 38 | OEt | OMe | 4-morpholin-2-yl-* | H | 425.1 |
| 39 | OEt | OMe | 4-morpholin-2-yl-* | H | 425.1 |
| 40 | OiPr | OMe | rac-4-morpholin-2-yl- | H | 439.6 |
| 41 | OiPr | OMe | 4-morpholin-2-yl-* | H | 439.1 |
| 42 | OiPr | OMe | 4-morpholin-2-yl-* | H | 439.1 |
| 43 | Propyl | OMe | rac-4-piperazin-2-yl- | H | 422 |
| 44 | Ethyl | OMe | 4-piperazin-2-yl-* | H | 408 |
| 45 | Ethyl | OMe | 4-piperazin-2-yl-* | H | 408 |
| 46 | OEt | OMe | 4-(R)-pyrrolidin-2-yl- | 2-Me | 423 |
| 47 | OiPr | OMe | 4-(R)-pyrrolidin-2-yl- | 2-Me | 437 |
| 48 | OEt | OMe | 4-(R)-pyrrolidin-2-yl- | 3-Me | 423 |
| 49 | OiPr | OMe | 4-(R)-pyrrolidin-2-yl- | 3-Me | 437 |
| 50 | OEt | OMe | 4-(R)-pyrrolidin-2-yl- | 3-Et | 437 |
| 51 | OiPr | OMe | 4-(R)-pyrrolidin-2-yl- | 3-Et | 451 |
| 52 | OiPr | OMe | 4-(R)-pyrrolidin-2-yl- | H | 423.8 |
| 53 | OEt | OMe | 4-piperazin-2-yl-* | H | 424.0 |
| 54 | OEt | OMe | 4-piperazin-2-yl-* | H | 424.0 |
| 55 | OiPr | OMe | 4-piperazin-2-yl-* | H | 438 |
| 56 | OiPr | OMe | 4-piperazin-2-yl-* | H | 438 |
| 57 | OEt | OMe | 4-(R)-pyrrolidin-2-yl- | 3-F | 427.8 |
| 58 | OiPr | OMe | 4-(R)-pyrrolidin-2-yl- | 3-F | 441.8 |
| 59 | OEt | OMe | rac-4-(2-Me-piperidin-2-yl)- | H | 437.2 |
| 60 | OiPr | OMe | 4-(R)-pyrrolidin-2-yl- | 2- | 453.17 |
| | | | | OMe | |
| 61 | OEt | OMe | 4-(R)-pyrrolidin-2-yl- | 2- | 439.18 |
| | | | | OMe | |
| 62 | Ethyl | OMe | 4-(R)-pyrrolidin-2-yl- | 2-Me | 407.2 |
| 63 | Ethyl | OMe | 4-(R)-pyrrolidin-2-yl- | 3-F | 411.17 |
| 64 | OiPr | OMe | 4-(R)-pyrrolidin-2-yl- | 2-F | 441.18 |
| 65 | OEt | OMe | 4-(R)-pyrrolidin-2-yl- | 2-F | 427.19 |
| 66 | OEt | OMe | 4-(R)-N(Me)pyrrolidin-2-yl- | 3-Me | 437.2 |
| 67 | OiPr | OMe | 4-(R)-N(Me)pyrrolidin-2-yl- | 3-Me | 451.3 |
| 68 | Ethyl | OMe | 4-(R)-N(Me)pyrrolidin-2-yl- | 3-Me | 421.3 |
| 69 | Ethyl | OMe | 4-(R)-pyrrolidin-2-yl- | 3-Me | 407.22 |
| 70 | Ethyl | OMe | 4-(R)-pyrrolidin-2-yl- | 2-F | 411.17 |
| 71 | Methyl | OMe | 4-(R)-pyrrolidin-2-yl- | 3-Me | 393.21 |
| 72 | Methyl | OMe | 4-(R)-pyrrolidin-2-yl- | 3-F | 397.16 |
| 73 | OEt | OMe | 4-(R)-N(Et)pyrrolidin-2-yl- | 3-Me | 451.2 |
| 74 | OiPr | OMe | 4-(R)-N(Et)pyrrolidin-2-yl- | 3-Me | 465.2 |
| 75 | Methyl | OMe | 4-(R)-pyrrolidin-2-yl- | H | 379.18 |
| 76 | Methyl | OMe | 4-(R)-pyrrolidin-2-yl- | 2-F | 397.18 |
| 77 | Methyl | OMe | 4-(R)-pyrrolidin-2-yl- | 2-Me | 393.21 |
| 78 | Propyl | (S)OCH(Me)Et | 4-(R)-pyrrolidin-2-yl- | H | 449.1 |
| 79 | Propyl | (S)OCH(Me)Et | 4-(R)-pyrrolidin-2-yl- | 3-Et | 477.1 |
| 80 | Propyl | (S)OCH(Me)Et | 4-(R)-pyrrolidin-2-yl- | 2-Me | 463.1 |
| 81 | OiPr | OiPr | 4-(R)-pyrrolidin-2-yl- | H | 451.6 |
| 82 | OiPr | OiPr | 4-(R)-pyrrolidin-2-yl- | 2-Me | 465.2 |
| 83 | Propyl | (S)OCH(Me)Et | 4-(R)-pyrrolidin-2-yl- | 3-Me | 463.3 |
| 84 | Propyl | EtO | 4-(R)-pyrrolidin-2-yl- | H | 421.2 |
| 85 | Propyl | EtO | 4-(R)-pyrrolidin-2-yl- | 2-Me | 435.3 |
| 86 | Propyl | EtO | 4-(R)-pyrrolidin-2-yl- | 3-Me | 435.3 |
| 87 | EtO | EtO | 4-(R)-pyrrolidin-2-yl- | H | 423.2 |
| 88 | EtO | EtO | 4-(R)-pyrrolidin-2-yl- | 2-Me | 437.3 |
| 89 | EtO | EtO | 4-(R)-pyrrolidin-2-yl- | 2-F | 441.5 |
| 90 | OiPr | OMe | 4-(S)-morpholin-3-yl- | 3-Me | 453.3 |
| 91 | OiPr | OMe | 4(-2-Me-piperidin-2-yl)-* | H | 451.3 |
| 92 | OiPr | OMe | 4(-2-Me-piperidin-2-yl)-* | H | 451.3 |
| 93 | EtO | OMe | 4(-2-Me-piperidin-2-yl)-* | H | 437.3 |
| 94 | EtO | OMe | 4(-2-Me-piperidin-2-yl)-* | H | 437.3 |
| 95 | EtO | OMe | 3-(S)-morpholin-3-yl- | H | 425.3 |
| 96 | OiPr | OMe | 3-(S)-morpholin-3-yl- | H | 439.3 |
| 97 | Methyl | OMe | 4-(S)-morpholin-3-yl- | H | 395.1 |
| 98 | CH₂OMe | OMe | 4-(S)-morpholin-3-yl- | H | 425.2 |
| 99 | EtO | EtO | 4-(S)-morpholin-3-yl- | H | 439.2 |
| 100 | OiPr | OiPr | 4-(S)-morpholin-3-yl- | H | 467.2 |
| 101 | EtO | CH₂OMe | 4-(S)-morpholin-3-yl- | H | 439.3 |
| 102 | CH₂OMe | EtO | 4-(R)-pyrrolidin-2-yl- | H | 409 |
| 103 | EtO | CH₂OMe | 4-(R)-pyrrolidin-2-yl- | H | 423.4 |
| 104 | OiPr | OiPr | 4-(R)-pyrrolidin-2-yl- | 2-F | 469.5 |
| 105 | OiPr | EtO | 4-(R)-pyrrolidin-2-yl- | H | 437.3 |
| 106 | OiPr | EtO | 4-(S)-morpholin-3-yl- | H | 453.3 |

Bibliographic Conventions in above table:
*denotes a chiral compound with unknown absolute configuration
rac denotes a racemic mixture of the S and the R enantiomer

The compounds of formula I in free form or in pharmaceutically acceptable salt form, exhibit valuable pharmacological properties, e.g. as S1P1 receptor agonists, e.g. as indicated in in vitro and in vivo tests and are therefore indicated for therapy.

A. In vitro

The compounds of formula I have binding affinity to individual human S1P receptors as determined in following assays:

### A.1 In Vitro: GPCR Activation Assay Measuring GTP [γ-³⁵S] Binding to Membranes Prepared From CHO Cells Expressing Human EDG Receptors

S1P₁ (EDG-1) GTP [γ-³⁵S] binding assay: Homogenized membranes are prepared from CHO cell clones stably expressing a human EDG-1 N-terminal c-myc tag. Cells are grown in suspension in two 850 cm² roller bottles for three or fours days before harvesting. The cells are centrifuged down, washed once with cold PBS, and resuspended in ≦20 ml of Buffer A (20 mM HEPES, pH 7.4, 10 mM EDTA, EDTA-free complete protease inhibitor cocktail [1 tablet/25 ml]). The cell suspension is homogenized on ice, using a Polytron homogenizer at 30000 rpm at three intervals of 15 seconds each. The homogenate is first centrifuged at 2000 rpm on a tabletop low speed centrifuge for 10 minutes. The supernatant, after passing through a cell strainer, is then re-centrifuged at 50,000×g for 25 minutes at 4° C. The pellet is resuspended into buffer B (15% glycerol, 20 mM HEPES, pH 7.4, 0.1 mM EDTA, EDTA-free complete protease inhibitor cocktail [1 tablet/10 ml]). Protein concentration of the preparation is determined using the BCA Protein Assay kit (Pierce) using BSA as standard. The membranes are aliquoted and kept frozen at −80° C.

Solutions of test compounds ranging from 10 mM to 0.01 nM are prepared in DMSO. S1P is diluted in 4% BSA solution as positive controls. The desired amount of membrane preparation is diluted with ice-cold assay buffer (20 mM HEPES, pH 7.4, 100 mM NaCl, 10 mM MgCl₂, 0.1% Fatty acid-free BSA, 5 μM GDP) and vortexed well. 2 μl or less of compound is distributed into each well of a round-bottom 96-well polystyrene assay plate, followed by addition of 100 μl of diluted membranes (3-10 μg/well) and kept on ice until the addition of hot GTPγS. [³⁵S]-GTPγS is diluted 1:1000 (v/v) with cold assay buffer and 100 μl is added into each well. The reaction is carried out at room temperature for 90 minutes before the membranes are harvested onto Perkin-Elmer Unifilter® GF/B-96 filter plate using a Packard Filtermate Harvester. After several washes with wash buffer (20 mM HEPES, pH 7.4, 100 mM NaCl, 10 mM MgCl₂), and a rinse with 95% ethanol, the filter is dried in a 37° C. oven for 30 minutes. MicroScint-20 is added and the plate sealed for scintillation counting on TopCount. EC50 values are obtained by fitting the GTP [γ-³⁵S] binding curves (raw data) with the dose response curve-fitting tool of GraphPad Prism. Six or twelve different concentrations are used to generate a concentration response curve (using three data points per concentration).

S1P3,-5,-6 and -8 GTP [γ-³⁵S] binding assays are carried out in a comparable manner to the S1P1 GTP [γ-³⁵S] binding assay using membranes from CHO cells stably expressing c-terminal c-myc tagged or untagged receptors. For each membrane preparation, titration experiments are first run with S1P control to determine the optimal amount of membranes to be added per assay well.

Compounds of formula I are tested according to the above assay and typically exhibit selectivity for the S1P1 receptor, e.g. S1P1 receptors with an EC₅₀<1 μM.

Moreover, compounds of formula I may exhibit selectivity for the S1P1 receptor compared to S1P3, S1P4 and S1P5, e.g. may at least be 20 fold selective for S1P1 compared to S1P3, S1P4 and S1P5.

Also typically, compounds of formula I may have a so-called dual selectivity for the S1P1 and S1P5 receptor over the other subtypes, namely S1P3 and S1P4. Said selectivity is typically around 20-30 (in terms of receptor affinity). Such dual S1P1/S1P5 receptor agonists have also valuable pharmacological efficacies.

| Ex | S1P-1 EC₅₀ [μM] |
|---|---|
| 1 | 0.0001 |
| 3 | 0.0016 |
| 8 | 0.0075 |
| 23 | 0.0755 |
| 27 | 0.0002 |
| 41 | 0.0029 |
| 43 | 0.0001 |
| 44 | 0.0002 |
| 53 | 0.0046 |
| 62 | 0.0005 |
| 72 | 0.0018 |
| 82 | 0.0024 |
| 89 | 0.0128 |
| 90 | 0.0062 |
| 96 | 0.0028 |

### A.2 FLIPR Calcium Flux Assay

Compounds of the invention are tested for agonist activity on S1P1, S1P3, S1P5, and S1P6 with a FLIPR calcium flux assay. Briefly, CHO cells expressing an S1P receptor are maintained in F-12K medium (ATCC), containing 5% FBS, with 500 ug/ml of G418. Prior to the assay, the cells are plated in 384 black clear bottom plates at the density of 10,000 cells/well/25 [symbol] l in the medium of F-12K containing 1% FBS. The second day, the cells are washed three times (25 μl/each) with washing buffer. About 25 μl of dye are added to each well and incubated for 1 hour at 37° C. and 5% CO₂. The cells are then washed four times with washing buffer (25 μl/each). The calcium flux is assayed after adding 25 μl of SEW2871 (published by Rosen et al., used as reference) solution to each well of cells. The same assay is performed with cells expressing each of the different S1P receptors. Titration in the FLIPR calcium flux assay is recorded over a 3-minute interval, and quantitated as maximal peak height percentage response relative to S1P-1 activation. The compounds of the invention are active in this assay at a concentration of from 10⁻¹² and 3.10⁻⁵ nM. For example example 13 as an EC₅₀ 92 nm for S1P-1 and an EC₅₀>1 um for all the other isoforms (S1P-2, S1P-3, S1P-4, S1P-5).

### B. In Vivo: Screening Assays for Measurement of Blood Lymphocyte Depletion

Measurement of circulating lymphocytes: Compounds to be tested are dissolved in DMSO/PEG200 and further diluted with deionized water. Rats (Lewis strain, female, 6-12 weeks old) are administered 1 mg/kg of compound to be tested in 4 ml/kg vehicle (max. 2% DMSO/max. 2% PEG200/water) via per os application. DMSO/PEG200/water and FTY720 (0.3 mg/kg) are included as negative and positive controls, respectively.

Blood is collected from the sublingual vein 2, 6, 24 and 48 hours after administration under short isoflurane anesthesia. Whole blood samples are subjected to hematology analysis. Peripheral lymphocyte counts are determined using an automated analyzer. Subpopulations of peripheral blood lymphocytes are stained by fluorochrome-conjugated specific antibodies and analyzed using a fluorescent activating cell sorter (Facscalibur). Two rats are used to assess the lymphocyte depletion activity of each compound screened. The result is an ED₅₀, which is defined as the effective dose required to display 50% of blood lymphocyte depletion. Compounds of formula I are tested according to the above assay and have an ED₅₀ of less than 10 mg/kg. For example compound of example 9 as an ED₅₀=1.4 mg/kg at 6 hours.

The compounds of formula I are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves opthalmopathy, alopecia areata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome or viral infections, e.g. AIDS, viral hepatitis, chronic bacterial infection, or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

In accordance with the foregoing the present invention further provides:
1.1 A method for preventing or treating disorders or diseases mediated by lymphocytes, e.g. such as indicated above, in a subject in need of such treatment, which method comprises administering to said subject an effective amount of a compound of formula I or a pharmaceutically acceptable salt thereof;
1.2 A method for preventing or treating acute or chronic transplant rejection or T-cell mediated inflammatory or autoimmune diseases, e.g. as indicated above, in a subject in need of such treatment, which method comprises administering to said subject an effective amount of a compound of formula I or a pharmaceutically acceptable salt thereof;
2. A compound of formula I, in free form or in a pharmaceutically acceptable salt form for use as a pharmaceutical, e.g. in any of the methods as indicated under 1.1 or 1.2 above.
3. A pharmaceutical composition, e.g. for use in any of the methods as in 1.1 or 1.2 above comprising a compound of formula I in free form or pharmaceutically acceptable salt form in association with a pharmaceutically acceptable diluent or carrier therefor.
4. A compound of formula I or a pharmaceutically acceptable salt thereof for use in the preparation of a pharmaceutical composition for use in any of the method as in 1.1 or 1.2 above.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g. a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CC1779, ABT578, AP23573, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

Where the compounds of formula I are administered in conjunction with other immunosuppressive/immunomodulatory, anti-inflammatory. chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth. In accordance with the foregoing the present invention provides in a yet further aspect:
5. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated above.
6. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula I 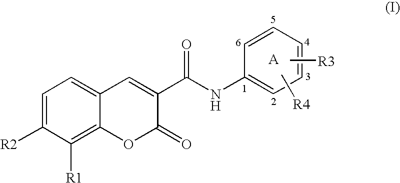
wherein
each of R₁ and R₂, independently, is selected from the group consisting of hydrogen; halogen; nitro; optionally substituted C₁₋₈alkyl; optionally substituted haloC₁₋₈alkyl; optionally substituted C₁₋₈alkoxy; and optionally substituted haloC₁₋₈alkoxy,
R₃ is a saturated heterocyclic ring comprising at least one ring N atom and which is attached to ring A through a ring C atom, this or any other ring C atom being optionally substituted;
R₃ is in position 3 or 4;
R₄ is hydrogen; hydroxyl; halogen; haloC₁₋₈alkyl; optionally substituted C₁₋₆alkyl; C₁₋₆alkoxy; or haloC₁₋₆alkoxy;
R₄ is in position 2 or 3;
or a physiologically hydrolysable derivative thereof, a salt, hydrate and/or solvate thereof.

2. The compound according to claim 1, wherein R₃ is a three to eight membered saturated, heterocyclic ring of formula (Ib) 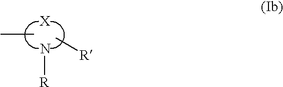
wherein X is selected from NR, O, CH₂ and S atom, wherein
R and R′ are independently from each other H or C₁₋₄ alkyl, and
wherein said R₃ is attached to said ring A in formula (I) via any carbon atom within the ring of formula (Ib) carrying a hydrogen atom, which hydrogen is then replaced by said attachment.

3. The compound of claim 1, wherein R₃ is a three to eight membered saturated, heterocyclic ring comprising 1 or 2 heteroatoms, said heteroatoms being selected from N, O and S.

4. (canceled)

5. The compound of claim 1, wherein the carbon atom of attachment is in position 2 or 3 with regard to the hetero-atom of R₃.

6. The compound of claim 1, wherein each of R₁ and R₂, independently, is selected from the group consisting of optionally substituted C₁₋₈alkyl; optionally substituted haloC₁₋₄alkyl; optionally substituted C₁₋₄alkoxy; optionally substituted haloC₁₋₈alkoxy.

7. The compound of claim 1, wherein each of R₁ and R₂, independently, is selected from the group consisting of C₁₋₈alkyl; and C₁₋₈alkoxy.

8. The compound of claim 1, wherein R₃ is 3-S-morpholinyl, 2-S-piperazinyl, 2-R-piperidinyl or 2-R-pyrrolidinyl; and R₃ is attached in position 4 of Ring A.

9-10. (canceled)

11. A method for treating disorders or diseases mediated by lymphocytes, in a subject in need of such treatment, which method comprises administering to said subject an effective amount of a compound of formula I or a pharmaceutically acceptable salt thereof.

12. The method of claim 11 where the disorders or diseases are selected from the group consisting of acute or chronic rejection of cell, tissue or organ allo- or xenografts or delayed graft function, graft versus host disease, autoimmune diseases, rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves opthalmopathy, alopecia areata and others, allergic diseases, allergic asthma, atopic dermatitis, allergic rhinitis/conjunctivitis, allergic contact dermatitis, inflammatory diseases optionally with underlying aberrant reactions, inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, myocardial infarction, stroke, gut ischemia, renal failure or hemorrhage shock, traumatic shock, cancer, breast cancer, T cell lymphomas or T cell leukemias, infectious diseases, toxic shock, septic shock, adult respiratory distress syndrome or viral infections, AIDS, viral hepatitis, chronic bacterial infection, and senile dementia.
